(19) United States
(12) Patent Application Publication
(10) Pub. No.: US 2006/0013871 A1
(43) Pub. Date: Jan. 19, 2006

(54) Intimate coating of ibuprofen with poloxamers to enhance aqueous dissolution

(76) Inventors: Larry L. Berger, Chadds Ford, PA (US); Madurai G. Ganesan, Newark, DE (US); Nutan Gangrade, Hockessin, DE (US); Henricus J. C. Gommeren, Hockessin, DE (US); Torrence J. Trout, West Chester, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/201,890

(22) Filed: Aug. 11, 2005

Related U.S. Application Data
(63) Continuation of application No. 11/009,416, filed on Dec. 10, 2004, now abandoned.
(60) Provisional application No. 60/531,315, filed on Dec. 19, 2003.

(51) Int. Cl. A61K 9/20; A61K 9/36

(52) U.S. Cl. 424/464; 424/480

(57) ABSTRACT

This invention provides methods for enhancing the dissolution rate of Ibuprofen. More particularly, the invention provides a process for enhancing the solubility of Ibuprofen by intimately coating Ibuprofen particles with a Poloxamer. The invention also provides for a composition comprising a solid dosage form of Ibuprofen, wherein the surfaces of Ibuprofen particles have been intimately coated with a Poloxamer; a composition comprising a fast onset, solid dosage form of Ibuprofen; and a composition comprising Ibuprofen particles intimately coated with a Poloxamer.

This application is a non provisional of and claims priority benefits from U.S. Provisional Patent Application Ser. No. 60/531,315, filed Dec. 19, 2003 and is incorporated by reference herein in its entirety.

### FIELD OF THE INVENTION

This invention relates to the field of pharmaceutical processing and formulation methodology, particularly to methods for enhancing or increasing the dissolution of Ibuprofen. More particularly, the invention relates to a process for enhancing the dissolution rate of Ibuprofen by intimately coating Ibuprofen particles with a Poloxamer.

### BACKGROUND OF THE INVENTION

The rate of delivery of a drug to target organs in the human body determines the onset of efficacy. For many drug therapies, it is desired to have fast onset of action. For analgesics, for example, pain-relief is obtained faster if an oral medicine reaches the systemic circulation quickly. Therefore, a fast-onset oral dosage form should rapidly release the medicine in a fashion that will facilitate instant absorption through the gastrointestinal tract into the blood. Because of its low solubility in the gastric medium at low pHs, the absorption rate of Ibuprofen is determined by its dissolution rate. Although Ibuprofen can be delivered in a soluble form as a liquid, the high dose needed for efficacy and chances of precipitation in the stomach environment may limit the absorption rate of the drug. This invention describes a solid dosage form that produces fast dissolution rate of Ibuprofen compared to currently marketed solid oral dosage forms.

### SUMMARY OF THE INVENTION

The invention provides a method of enhancing the aqueous dissolution of Ibuprofen comprising intimately coating Ibuprofen particles with a Poloxamer. Another aspect of the invention is a composition comprising a solid dosage form of Ibuprofen, wherein the surfaces of Ibuprofen particles have been intimately coated with a Poloxamer. A further aspect of the invention is a composition comprising a fast onset, solid dosage form of Ibuprofen. The invention also provides for a composition comprising Ibuprofen particles intimately coated with a Poloxamer. In preferred embodiments, the coating method comprises wet granulation, and the Poloxamer comprises at least one of Poloxamer 188 and Poloxamer 407.

### BRIEF DESCRIPTION OF THE DRAWINGS

For a more complete understanding of the present invention and for further advantages thereof, reference is now made to the following Detailed Description taken in conjunction with the accompanying Drawings in which:

FIG. 1 shows a graphical depiction comparing the rate of dissolution of Ibuprofen, micronized Ibuprofen, Ibuprofen intimately coated with Poloxamer 188 by wet-mixing, Ibuprofen coated with Poloxamer 188 by dry-mixing, and a commercial tablet of Ibuprofen. Dissolution Profiles of Ibuprofen (900 mL pH 5.8 Buffer, paddle @ 50 rpm, 37 C))

FIG. 2 shows a graphical depiction comparing the dissolution profiles of Ibuprofen tablets coated with Poloxamer 188 and uncoated Ibuprofen tablets. Dissolution Profiles (pH 5.8) of Ibuprofen Tablets with and without Poloxamer 188 (½″, 2-4 k P tablets).

FIG. 3 shows a graphical depiction comparing the dissolution profiles at pH 5.8 of tablets containing uncoated Ibuprofen and tablets containing Ibuprofen coated with Poloxamer 188.

FIG. 4 shows a graphical depiction comparing the dissolution profiles in 0.1 N HCl of tablets containing uncoated Ibuprofen and tablets containing Ibuprofen coated with Poloxamer 188.

### DETAILED DESCRIPTION OF THE INVENTION

Applicants specifically incorporate the entire content of all cited references in this disclosure. Where a range of numerical values is recited herein, unless otherwise stated, the range is intended to include the endpoints thereof, and all integers and fractions within the range. It is not intended that the scope of the invention be limited to the specific values recited when defining a range.

Surfactants are amphiphilic compounds. Hydrophilic surfactants facilitate wetting of hydrophobic drugs in an aqueous medium by reducing the interfacial tension. Depending on the aqueous solubility of the drug, this may result in subsequent dissolution of the drug in the aqueous medium. Surfactants are also known to increase solubility of poorly soluble drug compounds.

This invention describes use of a Poloxamer as a hydrophilic surfactant to increase the rate of aqueous dissolution of Ibuprofen from an oral tablet formulation. Ibuprofen and a Poloxamer are mixed using a wet-mixing process whereby the surfaces of Ibuprofen particles are intimately coated with Poloxamer. The term “intimately coated” means coating a Poloxamer onto Ibuprofen particles in the presence of water or an aqueous liquid, a non-aqueous liquid, a liquefied gas, a supercritical fluid, or in the form of a hot melt. The liquid in the solution or suspension can be aqueous or non-aqueous. Examples of aqueous liquids include, but are not limited to, water and/or buffer solutions. Examples of non-aqueous liquids include, but are not limited to, acetone, ethanol, and isopropanol.

The Poloxamer-coated Ibuprofen particles can then be processed further into a solid dosage form.

Preferably, the Ibuprofen particles have a median particle size of from about 0.5 μm to about 35 μm; the invention, however, is not limited to any particular Ibuprofen particle size range.

Any one of several known processes can be used to accomplish the intimate coating of Ibuprofen with a Poloxamer, including wet granulation, fluid-bed granulation, spray coating, fluid-bed coating, or the particle coating process as described in co-pending, co-owned application Serial No. PCT 03/25883, which is herein incorporated by reference. These processes will serve to intimately contact the surface of the Ibuprofen particles with a Poloxamer in the presence of a liquid.

For purposes of this invention, the term “Poloxamer” refers to block copolymers of ethylene oxide and propylene oxide, and includes compositions known by the trade names Pluronic® or Lutrol®. Preferred grades of Poloxamer are Poloxamer 188 and Poloxamer 407.

Another aspect of the invention is a fast onset, solid dosage form of Ibuprofen. The term “fast onset” as used herein means a solid dosage form that at 37° C. disintegrates or dissolves upon contact with the dissolution medium and rapidly releases a drug in about 2.5 minutes to reach not less than about 8 mg total in 900 ml at pH 1.0, and about 20 mg total in 900 ml at pH 5.8. More preferred is a solid dosage form that disintegrates or dissolves upon contact with the dissolution medium and rapidly releases a drug in about 2.5 minutes to reach not less than about 16 mg total in 900 ml at pH 1.0, and about 80 mg total in 900 ml at pH 5.8. Solid dosage forms include, but are not limited to, coated or uncoated swallowable or chewable tablets, dry powders in hard or soft gelatin capsules, and dry powders in individual or multiple use packages for reconstituted suspensions or sprinkles. Preferable solid dosage forms are coated or uncoated swallowable or chewable tablets. Suitable methods for manufacturing solid dosage forms are well known in the art.

Additionally, the solid dosage form can further comprise at least one excipient. Excipients include, but are not limited to, diluents (sometimes referred to as fillers) including, for example, microcrystalline cellulose, mannitol, lactose, calcium phosphate, dextrates, maltodextrin, starch, sucrose, and pregelatinized starch; disintegrants including, for example, crospovidone, sodium starch glycolate, croscarmellose sodium, starch, pregelatinized starch, and carboxymethylcellulose sodium; binders including, for example, starch, hydroxypropyl cellulose, hydroxypropylmethyl cellulose, pregelatinized starch, guar gum, alginic acid, acacia, carboxymethylcellulose sodium, and polyvinyl pyrrolidone; glidants including, for example, colloidal silican dioxide and talc; and lubricants/antiadherents including, for example, magnesium stearate, calcium stearate, stearic acid, sodium stearyl fumarate, glyceryl monostearate, hydrogenated vegetable oil, and talc.

The solid dosage forms may be used for any convenient dosage amount of Ibuprofen. Generally, the level of Ibuprofen may be increased or decreased according to the judgment of the physician, pharmacist, pharmaceutical scientist, or other person of skill in the art. The amount of the remaining non-active ingredients can be adjusted as needed.

### EXAMPLES

The present invention is further defined in the following Examples.

It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modifications of the invention to adapt it to various uses and conditions.

The meaning of abbreviations is as follows: “hr.” means hour(s); “min.” means minute(s); “μm” means micrometer(s) or micron(s); “nm” means nanometer(s); “ml” means milliliter(s); “g” means gram(s); “in.” means inch(es); “psig” means pounds per square inch, gauge; “Kp” means kilopond(s); “M” means molar; “rpm” means revolutions per minute, “N” means normal.

Sources of the compounds referred to: Ibuprofen, USP (BASF); Poloxamer 188 NF (Spectrum Chemical Company, Gardena, Calif.); mannitol, USP (Roquette America, Inc., Gurnee, Ill.); microcrystalline cellulose, NF (FMC Corp., Philadelphia, Pa.); croscarmellose sodium (FMC Corp., Philadelphia, Pa.); magnesium stearate (Mallinckrodt, St. Louis, Mo.); SEPR ceramic grinding beads from S. E. Firestone Assoc. (Russel Finex Inc., Charlotte, N.C.); CO₂ (MG Industries, Malvern, Pa.).

### EXAMPLE 1

Intimately Coating Ibuprofen with Poloxamer by Wet-Mixing: 4.5 g Ibuprofen, which had been screened through a 20 mesh sieve, was mixed with 0.5 g of Poloxamer 188 using a mortar and pestle with 0.5-2.0 ml water added in small portions until the material appeared well-granulated. The granulated composition was dried overnight at 40° C. The dried material was screened through a 20 mesh sieve and mixed with 7.5 g mannitol, 1.875 g microcrystalline cellulose, and 0.6 g croscarmellose sodium. Then, 0.075 g magnesium stearate that had been previously screened through 30 mesh sieve was added to the powder mixture and mixed again. The final blend was compressed with a manual press using ½ in. diameter flat-faced beveled-edge tooling to a hardness of 2-3 Kp.

### EXAMPLE 2

Dry-Mixed Ibuprofen and Poloxamer: 4.5 g Ibuprofen, which had been screened through a 20 mesh sieve, was blended with 0.5 g Poloxamer 188, 7.5 g mannitol, 1.875 g microcrystalline cellulose, and 0.6 g croscarmellose sodium, using a mortar and pestle. The resulting mixture was blended with 0.075 g magnesium stearate. The final blend was screened through a 30 mesh sieve and compressed with a manual press using ½ in. diameter flat-faced beveled-edge tooling to a hardness of 2-3 Kp.

### EXAMPLES 3 AND 4

Ibuprofen without Poloxamer: All the materials were screened before mixing. 5.0 g Ibuprofen or micronized Ibuprofen was blended with 7.5 g mannitol, 1.875 g microcrystalline cellulose, and 0.6 g croscarmellose sodium. The resulting mixture was blended with 0.075 g magnesium stearate. The final blend was compressed with a manual press using ½ in. diameter flat-faced beveled-edge tooling to a hardness of 2-3 Kp.

In vitro dissolution experiments were done using a USP apparatus 2 with paddle at 50 rpm and media at 37° C. Samples were analyzed using an UV spectrophotometer at 221 nm.

### RESULTS FOR EXAMPLES 1-4

Ibuprofen has pH-dependent solubility. It is almost insoluble at pH 1.0 and is soluble at pH 7.0. pHs of 1.0 and 5.8 were used in this study because of the pH-dependent solubility of Ibuprofen and to allow for comparison of dissolution rates of formulations to evaluate differences.

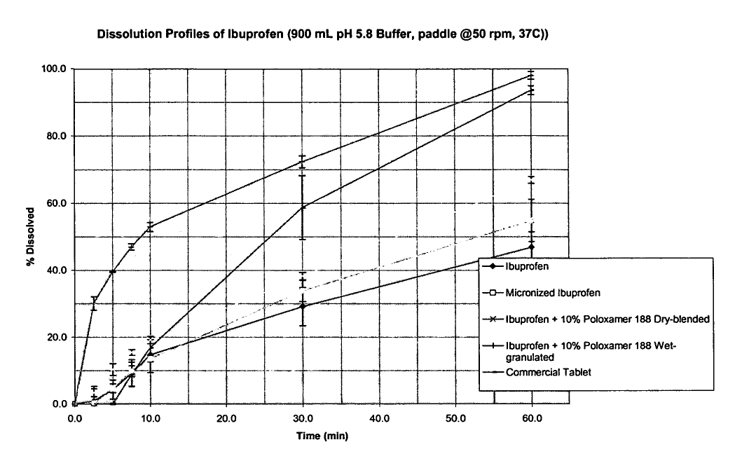

As shown in FIG. 1, dissolution results indicated that tablet compositions containing Ibuprofen intimately coated with Poloxamer by wet-mixing exhibit significantly faster dissolution profiles compared to tablets containing dry-mixed Ibuprofen and Poloxamer. Dry-mixing did not provide much faster dissolution than that seen from tablets containing Ibuprofen and tablets containing micronized Ibuprofen without any Poloxamer. For comparison, the dissolution profile of a commercial Ibuprofen product is also shown.

### EXAMPLE 5

Intimately Coating Ibuprofen with Poloxamer in the Presence of Supercritical CO₂: the Ibuprofen for this example was purchased from Spectrum Chemicals, lot number R00905. Particle size of feedstock was d16=21.8 micron, d50=41.21 microns and d84=71.05 microns, as measured in water suspension by Beckman Coulter LS230. A stirred high pressure jacketed vessel as described in co-owned PCT Publication WO 02/094443 was charged with 1,700 g of grinding beads (SEPR 0.8/1.0 mm), 100 g of Ibuprofen, and 2 g of Poloxamer 188 (Pluronic®) F68). The vessel was charged with 405 g of CO₂. The agitator rpm was 1,776. The mixing process was run for 1 hr. The final temperature and pressure were 28° C. and 1,434 psig, respectively.

Prior to the dissolution tests, 200 mg of the granulated Ibuprofen/Poloxamer mixture was mixed with 300 mg of mannitol (M300), 73 mg of Avicel PH102, 24 mg of Ac-Di-Sol, and 3 mg of magnesium stereate. The final blend was compressed with a manual press ½ in. diameter flat-faced beveled-edge tooling to a hardness of 3 Kp.

In vitro dissolution experiments were performed in 0.05 M phosphate buffer pH =5.8 with paddle stir at 50 rpm, 37° C.

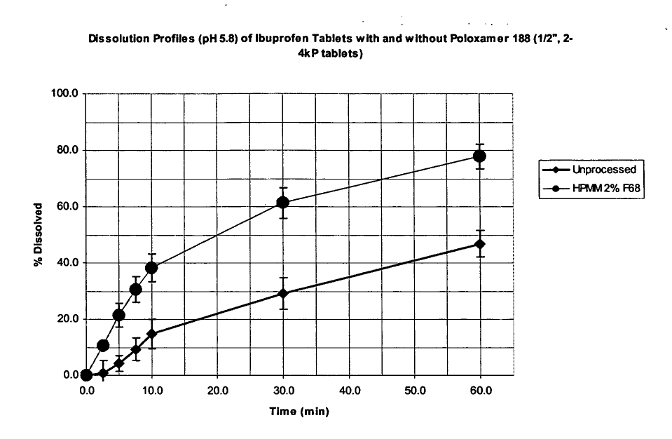

As shown in FIG. 2, dissolution results indicated that tablet compositions containing Ibuprofen intimately coated with Poloxamer in the presence of supercritical CO₂ exhibit a significantly faster dissolution profile compared to tablet compositions containing Ibuprofen without Poloxamer.

### EXAMPLE 6

Ibuprofen particles were coated using the apparatus and process as described in co-pending, co-owned application Serial No. PCT 03/25883 filed Aug. 14, 2003. The apparatus had a mixing chamber of either 2.54 cm in diameter and 19.05 cm long or 3.18 cm in diameter and 43.18 cm long with a nozzle throat of diameter between 0.64 cm and 1.02 cm and a central liquid feed tube diameter between 0.18 cm and 0.39 cm. The apparatus has a single screw metering feeder (AccuRate) for metering the solid particles which were delivered at 325-425 g/min. A peristaltic pump was fit with Masterflex LS/16 (3.1 mm I.D) Tygon elastomer tubing for metering the liquid. Ibuprofen was metered to the system (g/min.). Poloxamer 188 was dissolved in acetone to form a coating solution. The coating solution at room temperature was metered in a range of 20-30 g/min. to the nozzle. Heated nitrogen gas was used to atomize the coating solution, producing a negative pressure in the mixing zone to induce the addition of the Ibuprofen, and to provide the heat for evaporating any solvent from the Ibuprofen. The product of the mixing/drying was conveyed down a 1.25 in. (3.175 cm) I.D.×17 in. (17.78 cm) long tube to a cyclone to enable collection of the product. The product was passed repeatedly through the apparatus using the same process conditions as mentioned in this example. The final product samples had a Poloxamer mass fraction of 10-12% w/w.

Uncoated Ibuprofen was “pretreated” by passing it through the IT device without applying any coating. This breaks down the crystals to approximately 5-10 microns. At this size the Ibuprofen particles are mechanically stable during the IT coating process, so no new fresh surface area generation/particle breakdown occurs during further IT processing.

Particle size analysis was done in water suspension using Beckman LS230, however it is known that in an aqueous medium Poloxamer is likely to dissolve. Therefore, the results in Table 1 may reflect the initial size of the primary particles, that does not change after coating. D16, D50, and D84 represent sizes in micrometers based on cumulative volume distribution at 16%, 50%, and 84%, respectively.

TABLE 1
Particle Size Distribution of Ibuprofen Particles

| | D16 | D50 | D84 |
|---|---|---|---|
| Uncoated | 1.431 | 5.691 | 12.37 |
| Coated | 1.312 | 4.916 | 13.61 |

The uncoated and coated powders were directly-compressed separately into a 200 mg strength tablet after blending with mannitol, microcrystalline cellulose, croscarmellose sodium, and magnesium stearate. Powders were blended using a Turbula mixer (Glen Mills, Inc, Clifton, N.J.). The blend was compressed into tablets using a carver press (Carver Inc., Wabash, Ind.). The dissolution was performed in two dissolution mediums—0.1 N HCl and phosphate buffer (pH 5.8)—using a USP apparatus 2 at 50 rpm. As controls for comparison, unprocessed Ibuprofen, micronized Ibuprofen, and unprocessed Ibuprofen blended with Poloxamer were also formulated as tablets for dissolution studies.

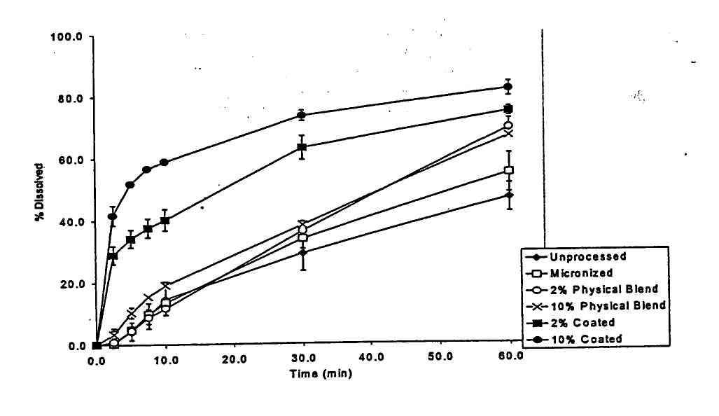

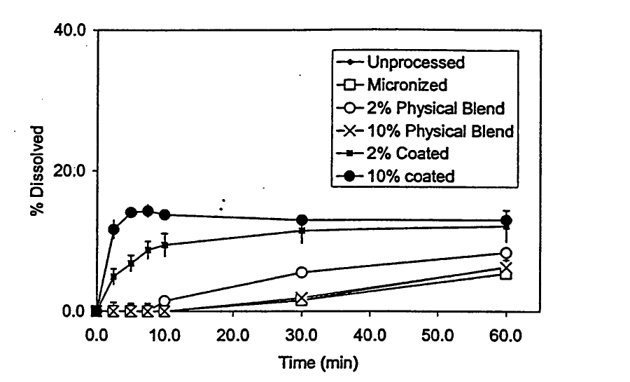

The results (FIGS. 3 and 4) show that at both pHs there is a significant increase in dissolution rate of Ibuprofen by coating compared to physical blending with approximately the same amounts of Poloxamer.

We claim:

1. A method of enhancing the aqueous dissolution of Ibuprofen comprising intimately coating Ibuprofen particles with a Poloxamer.

2. The method of claim 1, wherein the Poloxamer comprises at least one of Poloxamer 188 and Poloxamer 407.

3. The method of claim 1, wherein the coating is performed by wet-granulation.

4. The method of claim 1, wherein the Ibuprofen particles have a median particle size of from about 0.5 μm to about 35 μm.

5. A composition comprising a solid dosage form of Ibuprofen particles, wherein the surfaces of Ibuprofen particles have been intimately coated with a Poloxamer.

6. The composition of claim 5, wherein the Poloxamer comprises at least one of Poloxamer 188 and Poloxamer 407.

7. The composition of claim 5, wherein the Ibuprofen particles have a median particle size of from about 0.5 μm to about 35 μm.

8. The composition of claim 5, wherein the solid dosage form is selected from uncoated swallowable tablets, coated swallowable tablets, uncoated chewable tablets, and coated chewable tablets.

9. A composition comprising a fast onset, solid dosage form of Ibuprofen.

10. The composition of claim 9, wherein the fast onset, solid dosage form of Ibuprofen comprises particles that have been intimately coated with a Poloxamer.

11. The composition of claim 10, wherein the Poloxamer comprises at least one of Poloxamer 188 and Poloxamer 407.

12. The composition of claim 10, wherein the particles have a median particle size of from about 0.5 μm to about 35 μm.

13. The composition of claim 9, wherein the solid dosage form is selected from uncoated swallowable tablets, coated swallowable tablets, uncoated chewable tablets, and coated chewable tablets.

14. A composition comprising Ibuprofen particles intimately coated with a Poloxamer.

15. The composition of claim 14, wherein the Poloxamer comprises at least one of Poloxamer 188 and Poloxamer 407.

16. The composition of claim 14, wherein the Ibuprofen particles have a median particle size of from about 0.5 μm to about 35 μm.
